(19) United States
(12) Patent Application Publication
(10) Pub. No.: US 2016/0242694 A1
(43) Pub. Date: Aug. 25, 2016

(54) METHOD FOR DETECTING AROUSALS IN SLEEPING SUBJECTS

(71) Applicant: DYMEDIX CORPORATION, Shoreview, MN (US)

(72) Inventors: James P. Moore, Bloomington, MN (US); Todd M. Eiken, Lindstrom, MN (US)

(21) Appl. No.: 14/744,426

(22) Filed: Jun. 19, 2015

Related U.S. Application Data
(63) Continuation-in-part of application No. 14/631,188, filed on Feb. 25, 2015, now abandoned.

(51) Int. Cl. A61B 5/00; A61B 5/0255; A61B 5/0245

(52) U.S. Cl. CPC A61B 5/4809; A61B 5/0245; A61B 5/6826; A61B 5/6829; A61B 5/0255; A61B 5/4848; A61B 5/742

(57) ABSTRACT

A sensor for detecting sleep related autonomic nervous system arousals has a PVDF piezo component designed to be affixed to a body part exhibiting arterial pulse wave activity, such as a finger or toe. The signal from the PVDF piezo transducer due to pulse wave activity is signal processed and delivered to a PSG machine or a portable device for stowing digitized waveforms. It is found that the sensor output correlates well with EEG indications of cortical arousals.

### CROSS-REFERENCED TO RELATED APPLICATIONS

This application is a continuation-in-part of application Ser. No. 14/631,188, filed Feb. 25, 2015, entitled “METHOD FOR DETECTING AROUSALS IN SLEEPING SUBJECTS, which is deemed incorporated herein by reference in its entirety.

### STATEMENT REGARDING FEDERALLY SPONSORED RESEARCH OR DEVELOPMENT

Not applicable

### BACKGROUND OF THE INVENTION

I. Field of the Invention

This invention relates generally to diagnostic studies and, more particularly, to a new and simpler method for detecting episodes of sleep arousals.

II. Discussion of the Prior Art

As pointed out in a medical journal article entitled “Obstructive Sleep Apnea Syndrome: Effect Of Respiratory Events And Arousal On Pulse Wave Amplitude Measured By Photoplethysmography In NREM Sleep”, authored by Haba-Rubis et al. Sleep Health (2005) 9:73-81, sleep fragmentation caused by repetitive arousals have adverse consequences that contribute to the pathophysiology of sleep-disordered breathing. Thus, arousal frequency occasioned by upper airway resistance, episodes of sleep apnea or hypopnea, leg movement or other causes, is a parameter of interest in the conduct of sleep studies on patients. The American Academy of Sleep Medicine defines an arousal as a ten second interruption of brain waves.

Heretofore, the classical method for detecting arousals was to connect the subject to both an electroencephalogram (EEG) machine and an electromyograph (EMG) machine. To do so, a plurality of electrodes, usually 14, each with a lead for coupling the electrode to the EEG recorder machine, are affixed to the subject's head at predetermined locations for picking up cortical emanations and related muscle activity that are then graphically displayed on the EEG or a PSG machine. Interpretation of the graphical output requires personnel highly trained or experienced in neurology. Furthermore, placement of the sensing electrodes and maintaining their intimate contact with a subject's scalp and face during an extended period of sleep often proves difficult.

Another approach at detecting arousals involves the use of pulse oximetry that measures O₂ saturation in the blood using an IR light source that transmits through a finger to a photodiode. The signal output from the photodiode varies with the level of oxygen in the blood which, of course, varies upon cessation (apnea) or shallow breathing (hypopnea).

Because the skin is so richly perfused, it is relatively easy to detect the pulsatile component of the cardiac cycle. The DC component of the signal is attributable to the bulk absorption of the skin tissue, while the AC component is directly attributable to variation in blood volume in the skin caused by the pressure pulse of the cardiac cycle.

The height of the AC component of the photoplethysmogram is proportional to the pulse pressure, i.e., the difference between the systolic and diastolic pressure in the arteries,

Itamar Medical, Inc. of Caesarea, Israel, produces and sells a system under the trademark WatchPat that measures peripheral arterial tone in which a finger probe with a means for applying a static pressure to the finger sufficient to limit venous flow while permitting arterial blood flow such that an optical transducer or a Hall-Effect sensor within the probe can sense variations in size or volume of the digit. The device is further described in U.S. Pat. No. 6,461,305. It is also known that pulse wave amplitude changes correlate with epics of REM sleep. See the Goor et al U.S. Pat. No. 6,319,205.

In accordance with the present invention, a new method is offered for obtaining arousal information that generally reduces the complexity of the instrumentation involved. Rather than requiring a plurality of leads used to do a EEG workup, only a single pair of wires is needed.

### SUMMARY OF THE INVENTION

In carrying out the method of the present invention, there is provided a force-to-voltage transducer in the form of a strip of piezoelectric film, such as PVDF, that has metallic electrodes on its opposed major surfaces and a pair of electrical leads for connecting the electrode to a signal processing circuit that may be integrated with the leads. The output of the signal processing circuit can be connected directly to a conventional polysomnograph machine (PSG) for recording and display,

The PVDF film strip, when wrapped about a subject's finger, is exposed to effects of arterial blood flow such that the signals corresponding to pulse wave amplitude are generated. The pulse wave related signals are fed over the aforementioned leads and signal processing circuit for presentation on the display of the PSG or stored for later readout in a memory of a home sleep test (HST) recorder.

The signal processing circuit itself preferably comprises a filter network and peak detector that yields two signal channels, one providing a raw signal of individual pulse amplitudes and the other reflecting the change in pulse amplitude over time. It is found that arousals cause an identifiable change in the subject's pulse rate and amplitude and the rate and amplitude changes can be sensed by the PVDF film transducer.

### DESCRIPTION OF THE DRAWINGS

The foregoing features, objects and advantages of the invention will become apparent to those skilled in the art from the following detailed description of a preferred embodiment, especially when considered in conjunction with the accompanying drawings in which:

FIG. 1 is a block diagram of the apparatus used to carry out the method of the present invention;

FIG. 2 is an exploded drawing of the PVDF film transducer; and

FIG. 3 is a circuit for implementing the signal processor of FIG. 1;

FIG. 4 is a block diagram of a data acquisition module; and

FIG. 5 are waveforms representing the outputs from an EEG, a respiratory air flow sensor, an effort belt, a pulse oximeter and the raw and processed outputs from the signal processing circuit of FIG. 1.

### DESCRIPTION OF THE PREFERRED EMBODIMENTS

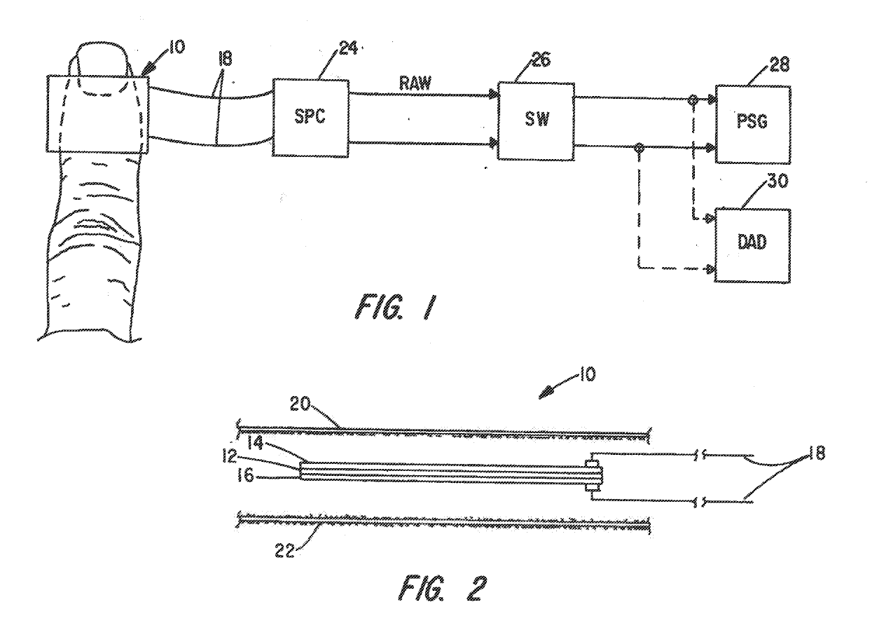

Referring first to FIG. 1, there is shown the apparatus for implementing the method of sensing arousal episodes of a person undergoing a sleep evaluation. It is seen to comprise a piezoelectric transducer 10, preferably comprising a polyvinylidene fluoride (PVDF) strip 12, as shown in the exploded view of FIG. 2. It has metallization layers 14 and 16 on opposed major surfaces thereof and to which are affixed electrical leads 18. The metalized PVDF film is sandwiched between adhesive tape strips 20 and 22 where adhesive tape strip 20 has adhesive only on the underside thereof, while adhesive strip 22 has adhesive on both major surfaces.

Referring again to FIG. 1, the transducer 10 is of a length and width allowing it to be wrapped about the distal end portion of a subject's finger such that the adhesive on the undersurface of the tape strip 22 will adhere the transducer 10 to the subject's finger.

The leads 18 connect to a signal processing circuit 24 which may conveniently be incorporated as an integral element of the leads. As will be further explained with the aid of FIG. 3, the signal processing circuit 24 incorporates a high-pass filter and a peak-detecting circuit such that the signal processing circuit 24 can be made to selectively output, on separate channels, a first waveform showing the transducer output voltage associated with each detected pulse wave and on a second channel, the variation in the peak voltages of the detected pulse wave.

The output from the signal processing circuit 24 is fed into a switch 26 whereby the raw signal on the first channel, the peak voltage on the second channel, or both, may be selectively fed to a polysomnograph machine 28 for display and analysis in those cases where the sensor module is being used on a patient in a sleep lab. The signals may alternatively be digitized and recorded in a memory forming part of a data acquisition device in a home sleep test (HST) recorder 30.

An implementation of a suitable signal processing circuit is shown in FIG. 3. The output of the transducer 10 is applied through a passive high-pass filter comprising capacitor C₁ and resistor R₁. The component values of this high-pass filter are chosen such that incoming frequencies lower than 10 Hz are attenuated.

The peak-detecting circuit includes an operational amplifier U₁ whose non-inverting input is coupled through a resistor R₂ and whose inverting input is directly connected to the output terminal of operational amplifier U₁. The output of C₁ is fed through diode D₁ to a holding capacitor C₂. The capacitor C₂ holds the current peak voltage. If a subsequent input voltage peak exceeds the current stored voltage, the OP-amp U₁ output goes positive until C₂ is charged up to the new peak value. If a subsequent input voltage is smaller, the diode D₁ prevents C₂ from discharging. The peak voltage detector can be reset by shorting out C₂ by closing the normally open switch. The Op-amp U₂ is a buffer for matching the impedance of peak detector to that of the PSG input.

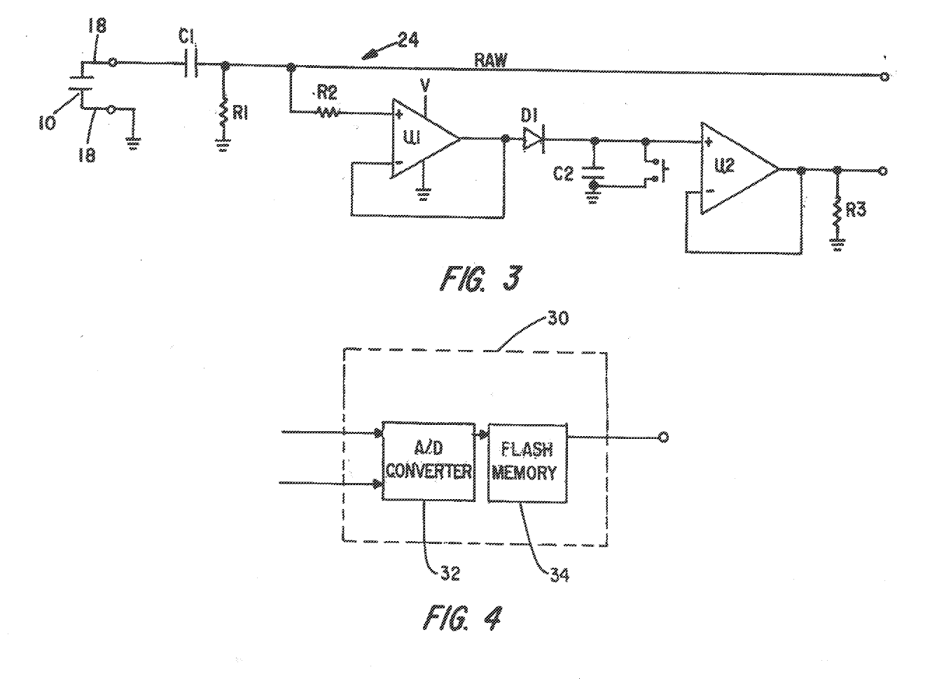

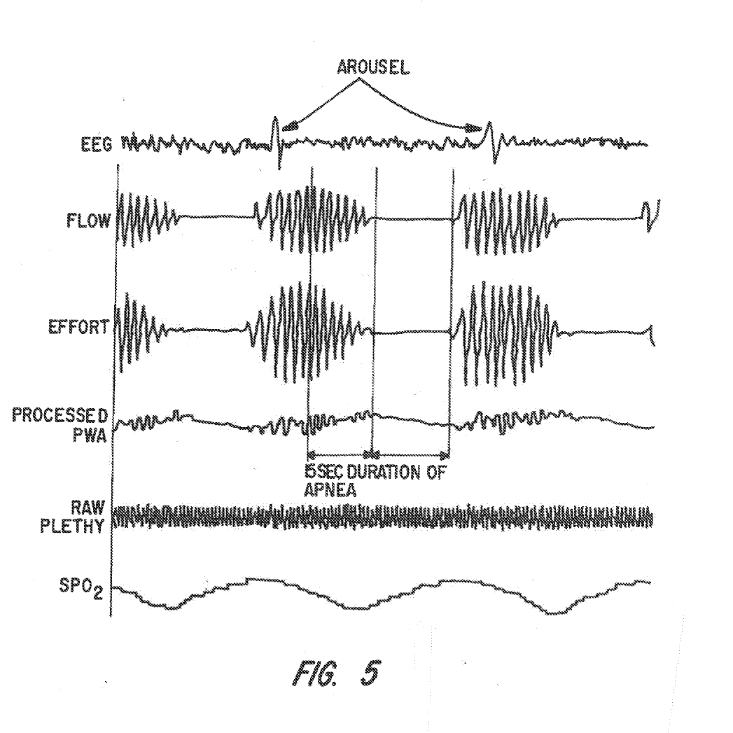

As mentioned, pulse wave amplitude variations may also be picked up by the PVDF sensor 10 and applied to a data acquisition device of a home sleep test monitor 30. As seen in FIG. 4, the home sleep test monitor 30 may comprise an analog to digital converter circuit 32 connected to the output terminals of the switch 26 and operative to convert the signal processed waveforms to a digital format which are then stored in a flash memory 34. Once the home sleep test is completed, the module 30 will be returned by the user to the module provider who will then download the memory to a computer for analysis and observation of arousal events.

Referring to the waveforms of FIG. 5, the one labeled EEG shows a typical output from a PSG machine resulting from an EEG input which heretofore has been the accepted apparatus for detecting cortical arousals. The waveforms labeled “Flow” and “EFFORT” are typical PSG outputs derived from a respiratory air flow sensor and from an abdominal or thoracic effort belt, each showing episodes of sleep apnea. Resumption of breathing following an apnea episode is due to an arousal. The blood oxygen saturation curve labeled SpO₂ is also seen to vary with breathing, falling during apnea episodes and rising following arousal and resumption of breathing.

Of interest here is how well the pulse wave amplitude (PWA) derived from the sensor 10 of FIG. 1 correlates with arousals detected using a “gold standard” EEG. Thus, the sensor of the present invention may be used in place of an EEG in a sleep test environments, thereby eliminating the need for multiple electrodes to be placed on the subject's head which tends to be uncomfortable, especially when the subject rolls over during sleep.

In that clinical studies have shown that children with sleep disorders are often inattentive or hyperactive, the present invention can find use in assessing ADHD (attention deficit/hyperactivity disorder) in children. Frequent arousals in nocturnal sleep may play a roll. Because the device of the present invention is a tool that detects sleep arousals, it can be used in diagnosis of ADHD to rule out sleep disruption.

It is further contemplated that the present invention can find use in assessing the effectiveness of certain dental appliances that are designed to eliminate upper airway obstruction and snoring by moving the lower jaw forward. These oral devices are not sold over-the-counter, but must be prescribed by a physician and fitted by a dentist or oral surgeon. By sensing arousals related to sleep disordered breathing during sleep following placement of the oral appliance, the need for adjustment (fine tuning) can be determined. The present invention provides a low cost method and apparatus for sensing sleep arousals.

A popular device for treating obstructive sleep apnea is a continuous positive airway pressure or CPAP system in which a source of pressurized air is delivered through a hose to a mask fitted over the nose and mouth of a subject to maintain the subject's airway open. More recently, an auto-adjust positive airway pressure (APAP) has become available in which the air pressure automatically adjusts to meet the subjects needs. It is contemplated that changes in pulse wave amplitude as sensed by a FVDF transducer strip affixed to the subject can be used to adjust the air pressure from the machine so as to minimize the occurrences of residual sleep arousals despite the elimination of apnea related events.

Also, in surgical procedures where the patient is given a general anesthetic, it is desirable that the anesthetist know of arousals so that the patient does not arrive at an awake state while the surgery is still in progress. The method and apparatus of the present invention may be used to sense arousals during general anesthesia and the anesthetic adjusted accordingly.

Also, in patients where it would be desirable to continuously monitor for and alert a patient of specific cardiac arrhythmias during the day or night (i.e. atrial fibrillation), the method and apparatus of the present invention may be used to sense arrhythmia related pauses in pulse rate and changes in pulse wave amplitude representing certain arrhythmia related blood volume changes. A portable hardware component could detect and alert the patient visually and audibly of the cardiac event.

This invention has been described herein in considerable detail in order to comply with the patent statutes and to provide those skilled in the art with the information needed to apply the novel principles and to construct and use such specialized components as are required, However, it is to be understood that the invention can be carried out by specifically different equipment and devices, including a more consumer focused model utilizing a smart phone. Also, various modifications, both as to the equipment and operating procedures, can be accomplished without departing from the scope of the invention itself.

1. A method for detecting periods of sleep arousal in a human subject comprising the steps of:
(a) appending a force-to-voltage transducer to the subject at a body location where pulse waves are detectable;
(b) connecting the transducer to inputs of a signal processing circuit for selectively generating a waveform showing peak voltages associated with each detected pulse wave and changes in pulse wave peak amplitude voltage; and
(c) connecting outputs of the signal processing circuit to one of a display device and a portable data acquisition device, wherein arousal periods are evidenced by changes in pulse wave voltage amplitude in the waveform.

2. The method of claim 1 wherein the force-to-voltage transducer comprises a strip of PVDF film having electrodes on opposed side surfaces of the strip.

3. The method of claim 2 wherein the PVDF film is affixed to the subject's phalanges.

4. The method of claim 1 wherein the display device comprises a PSG machine.

5. The method of claim 1 wherein the portable data acquisition device comprises an analog to digital converter coupled to receive outputs from the signal processing circuit and a memory module for storing outputs from the analog to digital converter.

6. The method of claim 1 wherein the detection of periods of arousal occur during the course of one of a sleep study, an ADHD assessment, and an evaluation of the effectiveness of a device designed to relieve airway obstruction and when a general anesthetic agent is being applied.
